(19) United States
(12) Patent Application Publication
(10) Pub. No.: US 2022/0128569 A1
(43) Pub. Date: Apr. 28, 2022

(54) PERIPHERAL BLOOD MARKER FOR CERABRAL HEMORRHAGE AND APPLICATION THEREOF

(71) Applicant: SHENZHEN PEOPLE'S HOSPITAL, Shenzhen (CN)

(72) Inventors: Zhijun HUANG, Shenzhen (CN); Jianjun LIU, Shenzhen (CN); Xifei YANG, Shenzhen (CN); Xiaoya HUANG, Shenzhen (CN); Shaorong QIU, Shenzhen (CN); Ruiping CAO, Shenzhen (CN); Xiaoli JIAN, Shenzhen (CN); Chengwen BAI, Shenzhen (CN)

(21) Appl. No.: 17/525,032

(22) Filed: Nov. 12, 2021

Related U.S. Application Data
(63) Continuation of application No. PCT/CN2019/097097, filed on Jul. 22, 2019.

(30) Foreign Application Priority Data
May 14, 2019 (CN) 201910398923.X

(51) Int. Cl. G01N 33/68

(52) U.S. Cl. CPC G01N 33/6842; G01N 2800/2871

(57) ABSTRACT

Disclosed are a peripheral blood marker for cerebral hemorrhage and an application thereof. The peripheral blood marker is a peripheral blood protein belonging to leucine-rich repeat (LRR) protein family. The peripheral blood protein is leucine-rich α2-glycoprotein-1 (LRG1). This disclosure further provides a method for diagnosing the cerebral hemorrhage.

### CROSS-REFERENCE TO RELATED APPLICATIONS

This application is a continuation of International Patent Application No. PCT/CN2019/097097, filed on Jul. 22, 2019, which claims the benefit of priority from Chinese Patent Application No. 201910398923.X, filed on May 14, 2019. The content of the aforementioned application, including any intervening amendments thereto, is incorporated herein by reference in its entirety.

### TECHNICAL FIELD

This application relates to medicines, and more particularly to a peripheral blood marker for cerebral hemorrhage and an application thereof.

### BACKGROUND

Cerebral hemorrhage is caused by non-traumatic rupture of blood vessels in the brain parenchyma, which accounts for 20-30% of all cerebral stroke cases, and has an acute mortality rate of 30-40%. There are diverse factors causing the cerebral hemorrhage, of which the blood vessel rupture from arteriosclerosis caused by hypertension is predominant. The cerebral hemorrhage is closely associated with hypertension, and almost 95% of cerebral hemorrhage cases have hypertension. The occurrence of cerebral hemorrhage is mainly related to a cerebrovascular lesion such as hyperlipidemia, diabetes, hypertension, aging of blood vessels and smoking behavior. The cerebral hemorrhage often occurs suddenly due to emotional agitation or exertion, leading to a high early mortality rate. Furthermore, most of survival cases develop a sequela such as dyskinesia, cognitive disorder, speech disorder, dysphagia, etc.

The common causes of the development of the cerebral hemorrhage include hypertension with arteriosclerosis, microaneurysm and capillary hemangioma, and moreover, it may also be derived from cerebral vascular malformation, dural arteriovenous malformation, cerebral amyloid angiopathy, cystic angiomatosis, cerebral venous thrombosis, specific arteritis, mycotic arteritis, moyamoya disease, anatomical variation of arteries, vasculitis and pituitary adenoma apoplexy.

In addition, there are some blood-related factors, such as anticoagulant, antiplatelet and thrombolytic therapy, Haemophilus infection, leukemia, thrombotic thrombocytopenia, intracranial tumor, alcoholism, sympathetic nerve stimulants, etc.

Exertion, climate change, unhealthy habits (such as smoking, alcohol abuse, excessive salt intake and overweight), fluctuation of blood pressure, emotional agitation, overwork, etc. are predisposing factors.

Hypertensive intracerebral hemorrhage usually occurs at an age of 50-70 years old, especially in males. It is easier to get onset in winter and spring as well as in activity and emotional agitation. Half of the patients suffer from a severe headache and emesis, and the elevated blood pressure. Clinical symptoms often reach the peak in a few minutes to a few hours, and vary with the location and amount of bleeding. Hemiparesis caused by hemorrhage of basal nucleus, thalamus and internal capsule is a frequent early symptom. A few cases suffer from focal seizure. Severe patients may become confused or unconscious quickly.

The dyskinesia is dominated by hemiplegia, and the speech disorder which mainly manifests as aphasia and slurred speech. About half of patients get emesis, which may be associated with intracranial hypertension, dizziness, and blood stimulation to the meninges during cerebral hemorrhage. The common symptom is drowsiness or coma, and the level is related to the location, amount and speed of cerebral hemorrhage. The case is prone to disorders of consciousness after suffering massive bleeding in a short period at a deep location. The case with cerebral hernia caused by intracranial hypertension may develop the anisocoria, as well as unilateral neglect and disorder of ocular movement. Patients with cerebral hemorrhage often stare at the bleeding side of the brain in the acute stage (referred to as gaze paralysis). Headache is the first symptom of the cerebral hemorrhage, and often occurs on the hemorrhage side. When the intracranial hypertension happens, the entire head may suffer from the pain. Dizziness is often accompanied by headache, especially during the cerebellum hemorrhage and brainstem hemorrhage.

In the case of a clear and definite diagnosis, the cerebrospinal fluid examination is generally not performed to prevent the cerebral hernia. However, when the brain computed tomography (CT) scan and magnetic resonance imaging (MRI) are not available, the lumbar puncture can still play a certain role in the diagnosis. After the cerebral hemorrhage, the intracranial pressure is generally higher due to an edema of brain tissues. 80% of patients have bloody or yellow cerebrospinal fluid within 6 hours after onset. The possibility of cerebral hemorrhage cannot be completely ruled out even the lumbar puncture cerebrospinal fluid is clear. A dehydration agent should be administered to lower the intracranial pressure before surgery. Lumbar puncture should be contraindicated when there may be intracranial hypertension or cerebral hernia.

An elevated white blood cell count may be observed in a blood routine examination of severe patients in the acute stage, and the patients may be also tested positive for the urine glucose and proteinuria. An elevated blood glucose level in the acute cerebral hemorrhage is caused by a stress response, and the elevated blood glucose level not only directly reflects a metabolic state, but reflects the severity of a disease. A higher blood glucose level is often accompanied with a higher incidence of complications such as stress ulcer, cerebral hernia, metabolic acidosis, azotemia, etc., as well as a worse prognosis.

Neuroimaging is commonly used in the diagnosis of cerebral hemorrhage, and mainly includes the following types.

CT scan of the brain can clearly show the hemorrhage location, the hemorrhage level, the hematoma shape, whether it has penetrated into the ventricle and whether there are low-density edema zone and space-occupying effect around the hematoma. The lesion generally is a round or oval uniform high-density area with a clear boundary. The lesion is usually a high-density cast when there is a large amount of hemorrhage in the ventricle, and the ventricle is enlarged. After 1 week, a ring enhancement appears around the hematoma. The hematoma shows a low density or cystic changes after absorption. Dynamic CT can be also used to evaluate the progress of hemorrhage.

MRI and magnetic resonance angiography (MRA) are superior to the CT in the detection of structural abnormalities, and hemorrhagic foci of the brainstem and the epencephalon, and the monitoring of the progress of cerebral hemorrhage. Unfortunately, for the diagnosis of the acute cerebral hemorrhage, the MRI and MRA are inferior to the CT.

Digital subtraction angiography (DSA) can detect cerebral aneurysm, arteriovenous malformation, moyamoya disease and vasculitis.

An electrocardiography (ECG) examination is another tool for the cerebral hemorrhage. Patients with cerebrovascular disease may have changes in the cardiac function and vascular function because of cerebral-cardiac syndrome or heart disease, such as (1) heart block, such as prolonged P-R interval, nodal rhythm and atrioventricular dissociation; (2) arrhythmia, such as atrial or ventricular premature contraction; (3)S-T segment ischemic changes, such as elevation, depression and changes of T wave; and (4) others such as electrocardiogram changes of pseudo myocardial infarction.

Transcranial doppler sonography (TCD) can help determine the intracranial hypertension and brain death. When the hematoma size is higher than 25 mL and the TCD result shows asymmetric change in intracranial hemodynamics, it indicates that the intracranial pressure is asymmetrical. Compared to the average blood flow rate, the pulsatility index plays a better role in reflecting the asymmetry of intracranial pressure.

The diagnosis is specifically performed as follows. When elderly patients get an onset suddenly when doing activities or being emotional, and symptoms of focal neurological deficit and intracranial hypertension symptoms such as headache and emesis rapidly develop, it should be considered whether the cerebral hemorrhage occurs. Combining with the brain CT, whether the cerebral hemorrhage occurs can be clearly determined. The diagnosis basis of the cerebral hemorrhage is introduced as follows: most of patients are over 50 years old with a long-term history of hypertension with arteriosclerosis; sudden onset of symptoms during physical activity or emotional agitation includes symptoms of focal neurological deficit and intracranial hypertension symptoms, such as headache and emesis; and symptoms of dyskinesia and intracranial hypertension; and there are neurological signs in physical examination. By virtue of the brain CT scan, it can be observed that the intracerebral hematoma presents in a high density. Moreover, the hematomas with a diameter greater than 1.5 cm can be accurately shown; the hemorrhage location, the hematoma size, whether the hemorrhage breaks into the ventricle, and whether there are cerebral edema and cerebral herniation can be determined. The diagnosis is confirmed based on the hemorrhage lesion on the CT scan. The diagnosis rate of the CT for the cerebral hemorrhage is almost 100%.

The prognosis of the cerebral hemorrhage is shown as follows. At an initial stage of the cerebral hemorrhage, especially within the first 24 hours, a re-hemorrhage is likely to occur, which is life-threatening. Therefore, much attention should be always paid to the changes. Cerebral hemorrhage is an acute cerebrovascular disease with high disability and fatality rate. It has been statistically reported that the fatality rate of the cerebral hemorrhage can reach up to 68%, and about ⅘ of the patients died within 4 days, and most of the rest also died within 2 weeks. Death in the acute stage is generally associated with a direct damage to the central nervous system caused by cerebral hemorrhage and intracranial hypertension, and the death after the acute stage is generally associated with pulmonary infection, kidney failure, stress ulcers, deep venous thrombosis and pulmonary embolism.

The relationship between the cerebral hemorrhage and proteomics is explained as follows. Protein is the functional executor of genes. The study of protein structure, location and interaction between proteins will provide a direct basis for elucidating the essence of life. Almost all physiological and pathological processes, as well as effects of drugs and environmental factors rely on proteins. Certainly, some proteins have experienced changes in structure and function prior to the occurrence of detectable symptoms. Therefore, it is of great significance to find key proteins and marker proteins of various diseases for the disease diagnosis, pathological exploration and drug screening.

Proteins that experience a change in the peripheral blood in the early stage of cerebral hemorrhage include proteins secreted by the liver and other organs, and proteins released into blood from damaged brain tissue after the blood-brain barrier is damaged. It has been discovered that some proteins, such as a leucine-rich repeat protein family including eight leucine-rich repeats (LRR), have a certain relationship with cerebral infarction. It has been demonstrated that most of the LRR proteins that are highly expressed in the nervous system are transmembrane proteins. They are mainly used as cell adhesion molecules or ligand binding proteins to participate in the formation of synapses, the growth and development of neurites, the transfer and release of neurotransmitters and other normal physiological activities of the nervous system.

Leucine-rich α2-glycoprotein-1 (LRG1) is a member of the LRR family. It has been proven that the LRG1 participates in important physiological and pathological processes in vivo such as protein interaction, signal transduction and cell adhesion. The LRG1 can be also expressed during the granulocyte differentiation. In recent years, researchers have found that the LRG is highly expressed in the serum of patients with liver cancer, lung cancer and pancreatic adenocarcinoma.

### SUMMARY

An object of this application is to provide a peripheral blood marker for cerebral hemorrhage and an application thereof.

The technical solutions of the present disclosure are described as follows.

In a first aspect, this application provides an application of a peripheral blood protein as a marker of cerebral hemorrhage, wherein the peripheral blood protein is a member of a leucine-rich repeat (LRR) protein family.

In some embodiments, the peripheral blood protein is leucine-rich α2-glycoprotein-1 (LRG1).

In a second aspect, this application provides an application of a reagent for quantifying a peripheral blood protein in the preparation of a diagnostic reagent for cerebral hemorrhage, wherein the peripheral blood protein is a member of a LRR protein family.

In some embodiments, the peripheral blood protein is LRG1.

In some embodiments, with respect to an expression level of the peripheral blood protein in a healthy subject or a subject with dizziness, syncope and coma that are not caused by cerebral hemorrhage, an up regulation in an expression level of the peripheral blood protein LRG1 indicates a risk of cerebral hemorrhage.

In some embodiments, the dizziness, syncope and coma that are not caused by cerebral hemorrhage, are caused by alcoholism, drug poisoning, or blood glucose disorder.

In some embodiments, the cerebral hemorrhage is acute cerebral hemorrhage.

In a third aspect, this application provides a method for diagnosing cerebral hemorrhage, comprising:

determining a content of a peripheral blood protein; and

predicting a risk of cerebral hemorrhage in a subject according to the content of the peripheral blood protein;

wherein the peripheral blood protein is a member of a LRR protein family.

In some embodiments, the peripheral blood protein is LRG1.

In some embodiments, with respect to an expression level of the peripheral blood protein in a healthy subject or a subject with dizziness, syncope and coma that are not caused by cerebral hemorrhage, an up regulation in an expression level of the peripheral blood protein indicates a risk of cerebral hemorrhage; and

the dizziness, syncope and coma that are not caused by cerebral hemorrhage, are caused by alcoholism, drug poisoning, or blood glucose disorder.

Compared to the prior art, the disclosure has the following beneficial effects.

The peripheral blood marker provided herein is suitable for the diagnosis of the cerebral hemorrhage, and can provide a reference for the clinical medication.

### DETAILED DESCRIPTION OF EMBODIMENTS

Technical solutions of this application will be described in detail below with reference to the experiments.

### Significance of Leucine-Rich α2-Glycoprotein-1 (LRG1) Concentration Change in Peripheral Blood in Diagnosis of Cerebral Hemorrhage

### Screening and Collection of Clinical Samples

90 cases with cerebral hemorrhage in the past 2 years were collected, all of which were the initial onset. The gender, age blood pressure, blood fat, blood glucose, liver function, kidney function, four blood coagulation items and electrocardiogram (ECG) of the patients were recorded. All of the patients were confirmed by computed tomography (CT) scan or brain magnetic resonance imaging (MRI). Meanwhile, normal cases with matching age and gender were taken as a control group. Samples were collected as follows. 5 mL of venous blood was collected from individual patients collected, allowed to stand for 15 min and centrifuged at 3000 r/min for 15 min to collect a serum, which was transferred to a sterile cryopreservation tube and stored at −80° C. Similarly, 5 mL of venous serum was collected from the control group and treated through the above steps. Moreover, venous serum samples were collected from 50 cases with coma or dizziness caused by alcoholism, drug poisoning and blood glucose disorder for differential diagnosis.

### Study on Plasma Samples of Patients

The serum and cells were separated with a high-speed centrifuge. Devices, materials and regents used herein included a homogenizer, a high-speed centrifuge, a 3 kDa ultrafiltration centrifuge tube and an inductively coupled plasma-mass spectrometry (ICP-MS). The water used herein was ultrapure water with a resistivity of 18.2 MΩ/cm. Pre-treatment of samples was performed as follows. The serum samples were taken out, thawed and determined for the LRG1 content at room temperature by an Elisa assay. Equipment used herein includes a fully automated biochemistry analyzers (DXC800, Beckman Coulter and AU400, Beckman Coulter), an electronic analytical balance (with a readability of 0.0001 g, Shimadzu), a microplate reader (imported), a Roche automatic electro-chemiluminescence immunoassay analyzer (Elecsys2010), a special protein analyzer (imported), a water purifier (MEDICA60), a hot water bath (DK-600), a low-temperature high-speed centrifuge (sigma), an ultralow-temperature refrigerator (Thermo Electron Corporation) and a low-temperature refrigerator (HFC350, Germany).

### Sample Analysis

The samples were analyzed by the Elisa assay.

### Statistical Analysis

The analysis results of individual groups were compared by T test using SPSS statistical software (P<0.05).

### Results

The LRG1 level was determined with kits respectively made in Japan and Wuhan. The detection value obtained by the kit made in Japan was much higher than that obtained by the kit made in Wuhan, which could be explained by that the antibody binding sites of these two kits were different. The normal range was determined by the mean value plus or minus 2 standard deviations, and an upper limit was the mean value plus 2 standard deviations. An upper limit (CUTOFF value) of the LRG1 kit made in Japan (LRG1 Japan) was 1300 ng/mL, and an upper limit (CUTOFF value) of the LRG1 kit made in Wuhan (LRG1 Wuhan) was 80 ng/mL. 88 cases with cerebral hemorrhage and 40 normal cases were detected with the Japan kit, where 82 of the 88 cerebral hemorrhage cases exceeded the CUTOFF value, and 8 of the 40 normal cases exceeded the CUTOFF value. 90 cases with cerebral hemorrhage and 60 normal cases were detected with the Wuhan kit, where 83 of the 90 cerebral hemorrhage cases exceeded the CUTOFF value (7 false negative cases), and 16 of the 60 normal cases exceeded the CUTOFF value (false positive rate: 26%).

The LRG1 content in the peripheral blood of the three groups was shown in Table 1.

TABLE 1
The LRG1 content in peripheral blood of three groups of cases
(X ± SD, with a unit of ng/mL)

| | LRG1 | LRG1 |
|---|---|---|
| | (Japan kit) | (Wuhan kit) |
| Normal people | 1183 ± 59 | 60 ± 11 |
| Patients with dizziness which is not caused | 1185 ± 80 | 46 ± 9 |
| by cerebral hemorrhage | | |
| Patients with acute cerebral hemorrhage | 1544 ± 68 | 145 ± 10 |

The LRG1 contents of the patients with acute cerebral hemorrhage were compared with the LRG1 contents of normal people and patients with a dizziness which was not caused by cerebral hemorrhage (P<0.01).

The sensitivity and specificity of LRG1 in the diagnosis of acute cerebral hemorrhage were shown in Table 2, where the sensitivity and specificity were calculated as follows:

sensitivity=the number of true positive cases/(the number of true positive cases+the number of false positive cases)×100%, namely, the accuracy for diagnosing the patients; and

specificity=the number of true negative cases/(the number of true negative cases+the number of false negative cases)×100%, namely, the accuracy for diagnosing the non-patients.

A false positive rate=the number of false positive cases/the number of gold standard negative cases.

A false negative rate=the number of false negative cases/the number of gold standard positive cases.

An upper limit (CUTOFF value) of a normal range determined by the LRG1 Japan kit was 1300 ng/mL, and an upper limit (CUTOFF value) of a normal range determined by the LRG1 Wuhan kit was 80 ng/mL. 88 cerebral hemorrhage cases and 40 normal cases were detected using the Japan kit, where 82 of the 88 cases exceeded the CUTOFF value, and 8 of the 40 normal cases exceeded the CUTOFF value. 90 cerebral hemorrhage cases and 60 normal cases were detected using the Wuhan kit, where 83 of the 90 cerebral hemorrhage cases exceeded the CUTOFF value, and 16 of the 60 normal cases exceeded the CUTOFF value (a false positive rate: 26%).

TABLE 2
Sensitivity and specificity of LRG1 in diagnosis of
acute cerebral hemorrhage

| | Positive | False | Negative | False |
|---|---|---|---|---|
| | coincidence rate | positive rate | coincidence rate | negative rate |
| LRG1 | 93.2% | 20% | 80% | 6.8% |
| (Japan kit) | | | | |
| LRG1 | 92.2% | 26% | 74% | 7.8% |
| (Wuhan | | | | |
| kit) | | | | |

TABLE 3
Concentration change of LRG1 in different stages of acute cerebral
hemorrhage (X ± SD, with an unit of ng/mL)

| | Normal | Time after onset of acute cerebral hemorrhage/ h | | | | |
|---|---|---|---|---|---|---|
| | people | 0.5 | 1 | 4 | 24 | 72 |
| LRG1 | 1183 ± | 1403 ± | 1886 ± | 1602 ± | 1512 ± | 1425 ± |
| (Japan | 59 | 85 | 121 | 82 | 113 | 105 |
| kit) | | | | | | |
| LRG1 | 60 ± | 173 ± | 182 ± | 133 ± | 123 ± | 120 ± |
| (Wuhan | 11 | 22 | 24 | 23 | 21 | 11 |
| kit) | | | | | | |

Compared with the normal people, the acute cerebral hemorrhage cases experienced a significant increase in the LRG1 concentration at 0.5 h and 72 hours after the onset (P<0.05).

What is claimed is:

1. A method for diagnosing cerebral hemorrhage, comprising:
determining a content of a peripheral blood protein; and
predicting a risk of cerebral hemorrhage in a subject according to the content of the peripheral blood protein;
wherein the peripheral blood protein is a member of a leucine-rich repeat (LRR) protein family.

2. The method of claim 1, wherein the peripheral blood protein is leucine-rich α2-glycoprotein-1 (LRG1).

3. The method of claim 1, wherein with respect to an expression level of the peripheral blood protein in a healthy subject or a subject with dizziness, syncope and coma that are not caused by cerebral hemorrhage, an up regulation in an expression level of the peripheral blood protein indicates a risk of cerebral hemorrhage; and
the dizziness, syncope and coma that are not caused by cerebral hemorrhage, are caused by alcoholism, drug poisoning, or blood glucose disorder.

4. The method of claim 2, wherein with respect to an expression level of the peripheral blood protein in a healthy subject or a subject with dizziness, syncope and coma that are not caused by cerebral hemorrhage, an up regulation in an expression level of the peripheral blood protein indicates a risk of cerebral hemorrhage; and
the dizziness, syncope and coma that are not caused by cerebral hemorrhage, are caused by alcoholism, drug poisoning, or blood glucose disorder.

5. The application of claim 1, wherein the cerebral hemorrhage is acute cerebral hemorrhage.
